(19) United States
(12) Patent Application Publication
(10) Pub. No.: US 2021/0186997 A1
(43) Pub. Date: Jun. 24, 2021

(54) METHODS AND COMPOSITIONS FOR INCREASING NAD LEVEL IN MAMMALS WITH D-RIBOSE AND VITAMIN B3

(71) Applicant: Bioenergy Life Science, Inc., Ham Lake, MN (US)

(72) Inventor: Yongquan Xue, Ham Lake, MN (US)

(21) Appl. No.: 17/054,512

(22) PCT Filed: May 10, 2019
(86) PCT No.: PCT/US2019/031889

§ 371 Date: Nov. 10, 2020

Related U.S. Application Data
(60) Provisional application No. 62/756,405, filed on Nov. 6, 2018.
(60) Provisional application No. 62/669,962, filed on May 10, 2018.

(51) Int. Cl. A61K 31/706; A61K 31/445; A61K 31/7004

(52) U.S. Cl. CPC A61K 31/706; A61K 31/7004; A61K 31/445

(57) ABSTRACT

Methods and compositions for increasing NAD levels in mammals by administering to the mammal an effective amount of D-ribose or D-ribose with Vitamin B3, where the Vitamin B3 may be niacin, nicotinamide, nicotinamide riboside or nicotinamide mononucleotide. The ratio between D-ribose and Vitamin B3 could vary between 0.5:10 and 10:0.5. The timing of administration to the mammal is at a time when the mammal is active or about to be active, whether the mammal is diurnal or nocturnal.

### BACKGROUND

Nicotinamide Adenine Dinucleotide (NAD) is pivotal for cell life; first as a reusable coenzyme for oxidation-reduction (redox) reactions and energy production by breaking down and converting nutrients into energy in the form of adenosine triphosphate (ATP); second as a consumable substrate in enzymatic reactions regulating crucial biological processes, including gene expression, DNA repair, cell death and lifespan, calcium signaling, glucose homeostasis, and circadian rhythms.

It has recently been discovered that NAD is the main substrate for three groups of proteins: (1) poly (ADP-ribose) polymerase (PARPs); (2) cADP-ribose synthase (CD38); and (3) Sirtuins (SIRT1-7). Each of these groups of proteins regulates a wide range of functions including DNA repair, mitochondrial dysfunction, neurodegeneration, and age-related metabolic disorder. For example, as coenzymes, NAD+ and its related metabolites, NADH, NADP+, and NADPH, participate in over 60% of reactions in cellular metabolism, and their homeostasis is the determinant for oxidation versus reduction and anabolism versus catabolism balances. As a consumable substrate, NAD concentration is directly linked with aging and fat composition. Additionally, NAD-consuming enzymes, including poly (ADP-ribose) polymerase (PARPs), Sirtuins (SIRT1-7), and cADP-ribose synthase (CD38), have wide spread ramification for health and disease. Therefore, NAD may serve as a therapeutic target for treating various metabolic or age-related conditions and promote health and longevity.

There are four NAD biosynthetic pathways operating in mammals, including a de novo pathway starting from amino acid tryptophan, and three alternative routes of pyridine salvage. These pyridines are, nicotinic acid (Na), nicotinamide (Nam), and nicotinamide riboside (NR), collectively referred to as vitamin B3, which may arise from dietary supply and/or intracellular NAD catabolism. The starting material for de novo pathway, tryptophan, is also from dietary protein sources such as egg, meat, and cheese.

Although all four NAD biosynthetic routes increase NAD levels in the body, the NAD generated via different routes are distributed differently in organs and tissues. In liver, all enzymes of the four pathways are known to be present, allowing conversion to NAD from all NAD precursors, and to re-fuel the whole organism with NAD through the bloodstream circulation. In other tissues, conversely, different enzyme levels reflect particular and intrinsic metabolic needs, also depending on the availability of exogenous pyridine source(s). Tryptophan is the only recognized source for de novo NAD synthesis, but it is generally considered insufficient to sustain normal NAD homeostasis. Most NAD in mammals is synthesized from Nicotinamide (Nam) via amidated salvage route. Liver again, with its elevated NAD turnover, represents a crucial tissue where Nam recycling prevails and NAD re-synthesis is regulated by nicotinamide phosphoribosyltransferase (NAMPT), also based upon a circadian transcriptional control by the clock machinery. Thus, it is understandable that NAD from Nam and NR has a distinctive local and temporal distribution than that from de novo synthesis.

Organ and tissue distribution of NAD metabolite is important for their biological function. A recent publication on NADPH variation between liver and muscle in exercising mouse is a prime example. While NAD deficiency has been linked with various pathological conditions, nicotinamide mononucleotide adenosyltransferase (NMNAT) gene alterations have been recently linked to cancer, Leber's congenital amaurosis, and axon protection in several neurodegeneration and acute injury models, including Wallerian degeneration models.

### DESCRIPTION OF THE DRAWINGS

FIG. 1 is a graph showing NAD levels measured in blood samples drawn from four groups of male rats at increments of 0.25, 0.5, 1, 2, 4, 8 and 24 hours after feeding each group different compounds of the same dosage; the first group being fed D-ribose, the second group being fed Niacin, the third group being fed D-ribose combined with Niacin, and the fourth group being fed Nicotinamide Riboside (NR).

FIG. 2 is a graph showing NR levels measured in the blood from each of the blood samples drawn at the increments identified in FIG. 1.

FIG. 3 is a graph showing NR levels measured in the blood of four groups of male and female rats at increments of 1, 2, 3, and 4 hours after being fed different concentrations of D-ribose combined with Nicotinamide (Nam).

FIG. 4 illustrates a proposed pathway for D-ribose combined with Nicotinamide (e.g., RiaGev™) and its metabolites.

FIG. 5 is a line chart showing NAD+ concentrations in blood after oral supplementation with D-ribose combined with Nicotinamide (e.g., RiaGev).

FIG. 6 is a line chart showing of NR concentrations in blood after oral supplementation with D-ribose combined with Nicotinamide (e.g., RiaGev).

FIG. 7 is a line chart showing of NMN concentrations in blood after oral supplementation with D-ribose combined with Nicotinamide (e.g., RiaGev).

FIG. 8 is a bar graph showing NAD+ distribution in liver, muscle and brain tissue after administration of different doses of RiaGev (D-ribose combined with Nicotinamide).

FIG. 9 is a bar graph showing NR distribution in liver, muscle, brain and adipose tissue after administration of different doses of RiaGev (D-ribose combined with Nicotinamide).

FIG. 10 is a bar graph showing NMN distribution in liver, muscle, brain and adipose tissue after administration of different doses of RiaGev (D-ribose combined with Nicotinamide).

FIG. 11 is a line chart showing dose-response relationships of RiaGev (D-ribose combined with Nicotinamide) and NAD+ in blood.

### DESCRIPTION

There are four pathways for NAD syntheses:

Pathway #1: salvage pathway of nicotinamide (Nam), represented as:

Nam+PRPP→NMN+ATP→NAD

Pathway #2: salvage pathway for nicotinic acid (Na), represented as:

Na+PRPP→NaMN+ATP→NaAD→NAD

Pathway #3: de novo biosynthetic pathway from amino acid tryptophan, represented as:

Tryptophin→NAD

Pathway #4: nicotinamide riboside (NR), represented as:

NR+ATP→NAD

Where:
Nam=Nicotinamide;
PRPP=phosphor-Ribose-pyrophosphate
NMN=Nicotinamide Mononucleotide;
NAD=Nicotinamide Adenine Dinucleotide
Na=Nicotinic Acid (Niacin)
PP=pyrophosphate

In three of the pathways (Pathways #1-#3), it is recognized that NAD levels in the body would be increased by supplying precursors or intermediates, such as Na, Nam, NR or NMN. Indeed, there are dietary supplement that contains these ingredients for NAD related enhancements.

In each of the three biosynthetic pathways (Pathways #1-3) and in the NR pathway (Pathway #4) PRPP and/or ATP are needed. It is known that both PRPP and ATP are extension products of D-ribose (i.e., D-ribose+ATP→PRPP). Therefore, Applicant hypothesized that it should be feasible to increase NAD levels in the body by administering D-ribose. Applicant is not aware of anyone previously attempting to increase NAD levels in the body by oral administration of D-ribose.

To test the hypothesis, Applicant designed experiments to test whether oral administration of D-ribose or D-ribose in combination with other compounds could increase NAD levels in the body of mammals, specifically Sprague-Dawley rats, and the conditions under which D-ribose alone or D-ribose in combination with other compounds could be utilized to improve NAD levels in the body of mammals.

### EXAMPLES

### Experiment 1:

Four groups of four male Sprague-Dawley rats were each bolus administered the different compounds identified in the table below at a dosage of 100 mg per kg of body weight:

TABLE 1

| | |
|---|---|
| Group 1 | 100 mg D-ribose per ml H2O |
| Group 2 | 100 mg Niacin per ml H2O |
| Group 3 | 100 mg D-ribose + Niacin per ml H2O |
| Group 4 | 100 mg Nicotinamide Riboside (NR) per ml H2O |

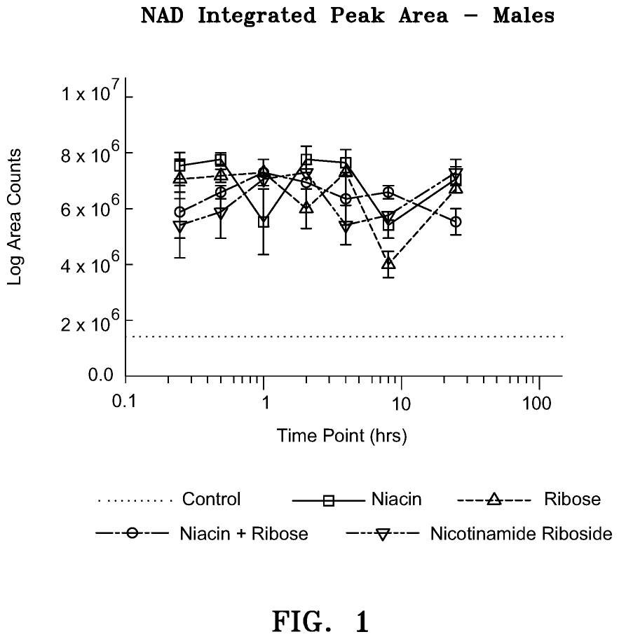

A blood sample of 0.1 ml was drawn from each rat of each group at increments of 0.25, 0.5, 1, 2, 4, 8 and 24 hours after feeding. Each of the blood samples were then measured by Liquid Chromatography/Mass Spectrometry (LC/MS) for NAD levels. The measured NAD levels are charted in FIG. 1, with each data point being the average of the four rats of each group.

Referring to FIG. 1, it is shown that each test group had significantly higher levels of NAD over the 24 hour period compared to the baseline (dashed line in FIG. 1, representing NAD level without testing compound administration). In particular, NAD levels for Group 3 (rats fed D-ribose+Niacin) increased after administration and stayed at the elevated level over longer period of time than the other groups. This is consistent with the biosynthetic mechanism of NAD from ribose and niacin. The positive control group (Group 4) had the highest peak value. However, the NAD level of Group 4 does not stay as steady as the NAD level of Group 3. Feeding D-ribose alone (i.e., the Group 1 rats) or feeding Niacin alone (i.e., the Group 2 rats) also resulted in elevated NAD levels consistent with biosynthetic mechanism. However, the NAD level of the Group 1 and Group 2 rats varies sharply, likely due to feeding or lighting time (discussed below).

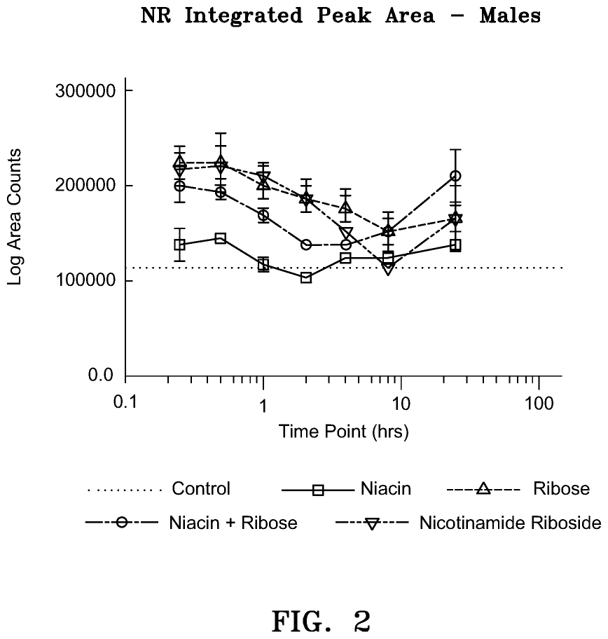

The blood drawn from each of the groups at the increments referenced above were also measured by LC/MS for NR levels. The measured NR levels are charted in FIG. 2, with each data point being the average of three animals of the same sex.

Referring to FIG. 2, the NR levels for each of the test groups is above the baseline (dashed line in FIG. 2), indicating that D-ribose, Niacin, or D-ribose combined with Niacin enhance NR levels in the rats. The overall trend of NR follows nicely with metabolic clock timing of NAD metabolites.

### Experiment 2:

Three groups of four male Sprague-Dawley rats were bolus administered three different doses of D-ribose combined with Nicotinamide (also known as Niacinamide) as identified in the table below:

TABLE 2

| | Nicotinamide + Ribose dosage |
|---|---|
| Group 1 | (100:180 w/w), 100 mg/ml H2O |
| Group 2 | (100:180 w/w), 300 mg/ml H2O |
| Group 3 | (100:180 w/w), 1000 mg/ml H2O |

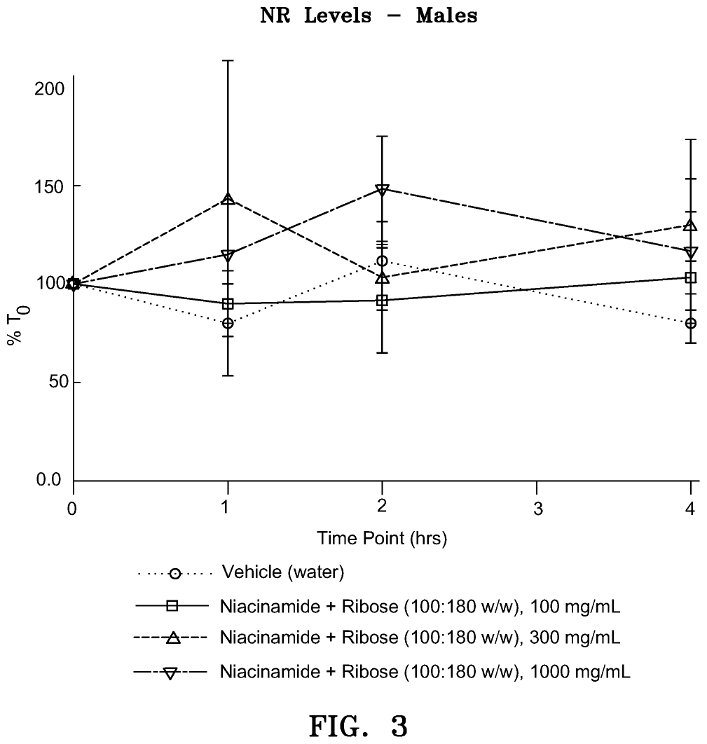

A blood sample of 0.1 ml was drawn from each rat at increments of 1, 2, 3 and 4 hours after feeding. Each of the blood samples were then measured by LC/MS for NAD and NR levels. The measured NR levels are charted in FIG. 3, with each data point being the average of the four the four rats from each group.

Referring to FIG. 3, it is shown that a dose of 300 mg/kg of body weight of Nicotinamide+Ribose is the optimal dose for the rat. Further increasing the dose level to 1000 mg/kg did not significantly enhance NR level. A dose of 100 mg/kg dose is not effective for NR level elevation, which is a little surprising since 100 mg/kg of D-ribose+Niacin demonstrated a good result in Experiment 1 as shown in FIG. 2.

Overall, the experiments demonstrate D-ribose alone, or D-ribose combined with Vitamin B3 (Niacin or Nicotinamide) increases NAD levels in mammals. However, Applicant determined that the exact dose scheme including optimal dose and timing needed to be refined in future experiments because the supplementation timing and sampling times seemed to play a big role in the experimental results.

For example, in Experiment 2, as shown in FIG. 2, the experiment was conducted only over a four-hour period and blood samples were drawn at 1, 2, 3 and 4 hours after supplementation in the morning. This time of supplementation and sampling did not result in meaningful results. Whereas, in Experiment 1, the experiment was conducted over a 24 hour period. The NAD levels are higher during night time hours only.

Applicant considered that evening feeding hours (between 8 and 24 hrs in FIG. 2) may be a better timing window for D-ribose+Vitamin B3 (Niacin or Nicotinamide) supplementation for rats than the morning hours because the morning hours fall into the trend of NAD decline in rats as shown in the NAD metabolic clock, because rats are nocturnal (discussed in more detail below). During this period, the amount of enzymes in NAD biosynthetic pathway are much less than during the feeding hours in the evening when NAD metabolite naturally increases for rats and other nocturnal animals.

To test this hypothesis, Applicant conducted additional experiments during evening feeding hours using a lower D-ribose+Vitamin B3 dose and with adjustments to the ratio between D-ribose and Vitamin B3 for the new dosing scheme. In conducting these additional experiments, Applicant utilized an optimized and fixed ratio (based on previous experiments) of nicotinamide and D-ribose, by using a product known as RiaGev™, to determine the pharmacodynamics and tissue distribution of NAD metabolites. RiaGev is available from Bioenergy Life Science, Inc., 13840 Johnson Street NE, Ham Lake, Minn. USA 55304.

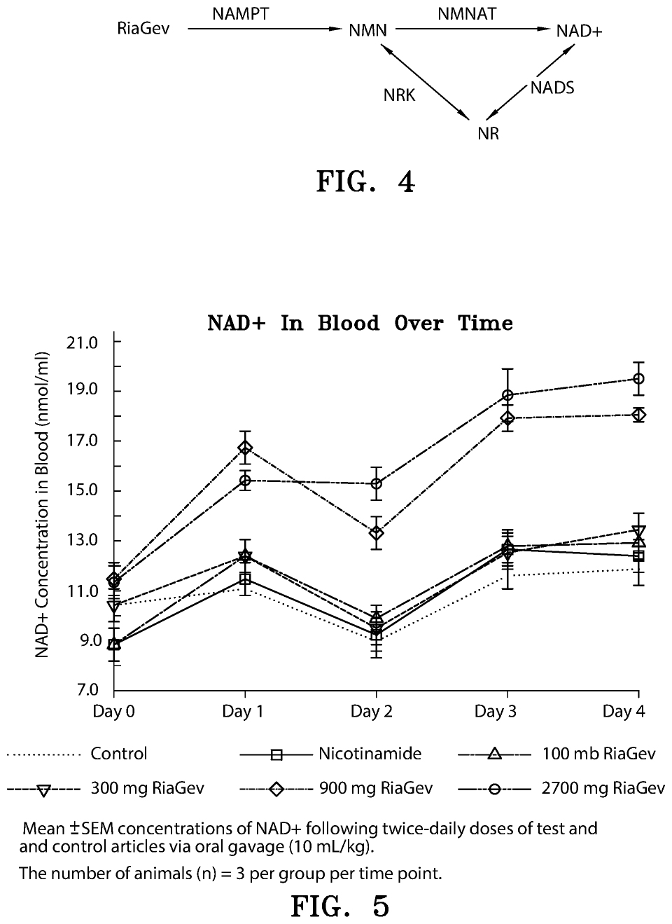

Metabolic analysis predicts that RiaGev supplementation would increase NAD+, NMN, and NR levels via the nicotinamide salvage pathway. As shown in FIG. 4, RiaGev is primarily converted to NMN by nicotinamide phosphoribosyltransferase (NAMPT), which then is converted to NAD+ by NMN adenosylphosphortranferase (NMNAT). An overflow of NMN may lose its phosphate moiety, generating NR, which is then converted into NAD in a series of steps—NRK: NR kinase, NADS: NAD synthase.

An experiment was designed to measure niacinamide/ribose metabolites in rat blood for each of three dose levels orally administered twice daily for five days.

### Experiment 3

Eighteen Sprague-Dawley male rats were selected as test animals in the study, with three rats for each of six treatment groups. Sprague-Dawley male rats were selected because they have been widely used in similar experiments with reliable results. The use of three rats per group offers the minimum number required to calculate useful means and standard deviations for graphical representation of the data.

For five consecutive days, the rats were dosed at approximately 7:30-8:30 a.m. and at approximately 4:30-5:45 p.m. Blood was collected approximately 0.5 to 1.5 hours following each p.m. dosing and immediately processed and assayed for NAD+, NMN, and NR by LC/MS/MS. The timing of the supplementation doses was chosen to mimic behavior in humans and other non-nocturnal mammals (i.e., diurnal mammals) of eating breakfast in the morning and dinner in the early evening by administering the doses to the animals at around those times. The sampling time was chosen to correspond to the times when NAD metabolite naturally increases in rats and other nocturnal mammals (i.e., in the evening) when the nocturnal animals are typically most active or about to become active. For humans and other diurnal animals, NAD metabolite naturally increases in the mornings when diurnal mammals are typically most active or about to become active. Thus the evening sampling time for the rats would correspond to the morning for humans and other diurnal mammals.

The animals were humanely euthanized after the final blood draw and representative samples of the liver, biceps muscle, peripheral adipose tissue, and whole brain were collected, snap frozen, extracted, and assayed for NAD+, NMN, and NR by LC/MS/MS. Table 3 identifies the groups and dosing schedule.
Inclusion Criteria: During the five-day dosing period, all 18 rats survived and appeared outwardly healthy.
Blinding: All testing was performed in a blinded manner. None of the research staff involved in the study were aware of the group assignment of any of the rats they were testing. One staff member prepared the dose solutions, coded the syringes of solutions (e.g., 1-6), and created the blind (treatment key).
Group Assignment: The rats were assigned to treatment groups based on Day −1 body weights such that group means were approximately equal. The rats were ranked by body weight and treatments assigned randomly within stratified sub-groups according to the total number of treatment groups in the study.
Control Article: Vehicle (0.5% MC/0.1% Tween 80 in water); coated nicotinamide.
Test Article: 12% nicotinamide+D-ribose administered twice daily for five consecutive days by oral gavage at doses of 100, 300, 900 and 2700 mg/kg at dose volumes of 10 ml/kg.
Dosing: The volume of test or control article injected was 10 ml/kg. The rats were dosed in sequence based on animal number so that the distribution of treatment across a given set of animals was not predictable.

TABLE 3
Groups and Dosing Schedule

| | | | | Dose | | Day of |
|---|---|---|---|---|---|---|
| Group | | | Dose | Vol. | | Admin./ |
| # | Treatment | N | (mg/kg) | (mL/kg) | Route | Frequency |
| 1 | Vehicle | 10 | N.A. | 10 | PO | Days 0-4/ |
| | | | | | | BID |
| 2 | Coated | 10 | 108 | 10 | PO | Days 0-4/ |
| | Nicotinamide | | | | | BID |
| 3 | 12% Nicotinamide + | 10 | 100 | 10 | PO | Days 0-4/ |
| | D-ribose | | | | | BID |
| 4 | 12% Nicotinamide + | 10 | 300 | 10 | PO | Days 0-4/ |
| | D-ribose | | | | | BID |
| 5 | 12% Nicotinamide + | 10 | 900 | 10 | PO | Days 0-4/ |
| | D-ribose | | | | | BID |
| 6 | 12% Nicotinamide + | 10 | 2700 | 10 | PO | Days 0-4/ |
| | D-ribose | | | | | BID |

### Results

1. Pharmacodynamics of RiaGev (D-Ribose Combined with Nicotinamide).

The NAD levels in the blood over time are presented in FIG. 5. After RiaGev ingestion, NAD levels increased steadily at all dosages in a dose dependent manner. The NAD levels reach their plateau after Day 4 (8 doses) of supplementation.

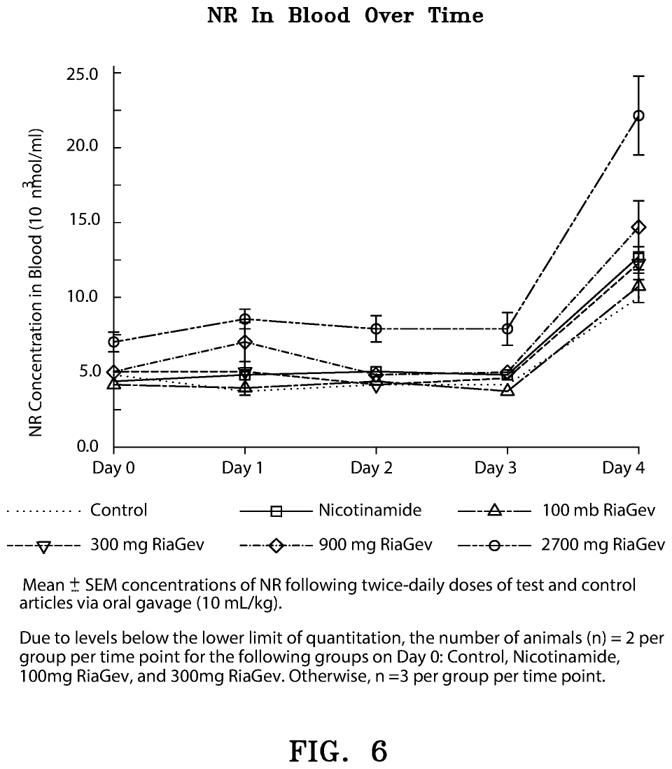

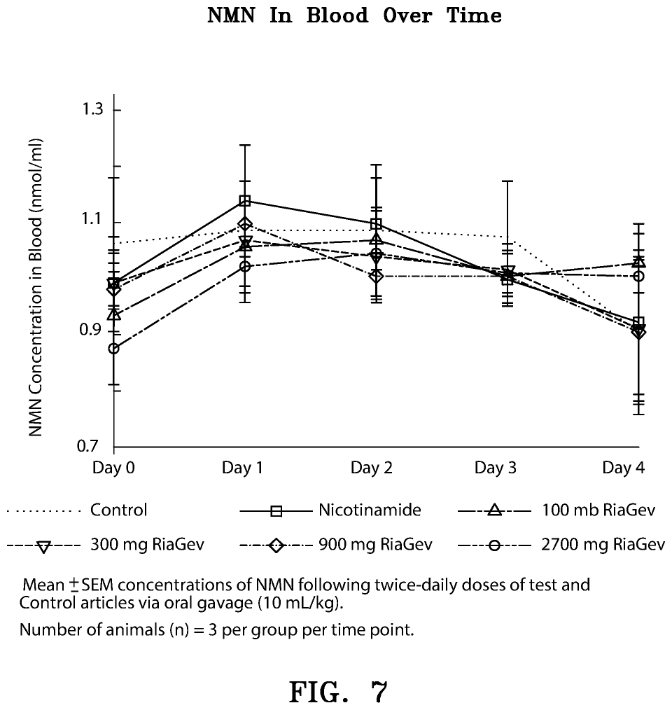

The NR levels in the blood over time are presented in FIG. 6. The NR measurement in blood also increased over time, although in a smaller scale. This is consistent with the fact the NR is an overflow shunt product in NAD biosynthesis (See FIG. 1). The NMN levels in the blood over time are presented in FIG. 7. NMN level in the blood does not significantly change, which is consistent with other publications.

### 2. Tissue Distribution of RiaGev Metabolites.

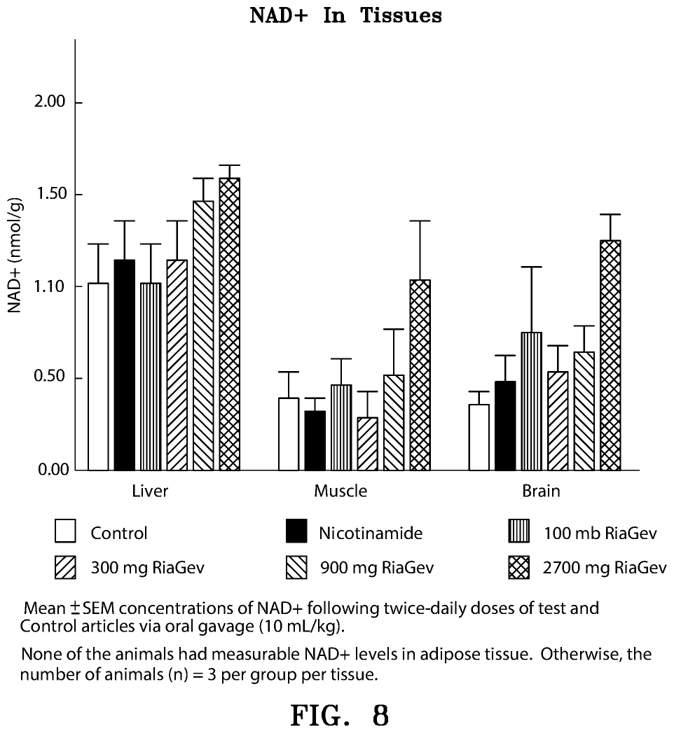

At the end of the five-day supplementation, tissues were harvested and analyzed for their NAD metabolites content. The NAD contents in the liver, muscle and brain are presented in FIG. 8. As shown in FIG. 8, the highest levels of RiaGev derived NAD were detected in the liver. The liver also has the highest level of background NAD. The brain is the second organ for NAD pool and muscle follows closely behind.

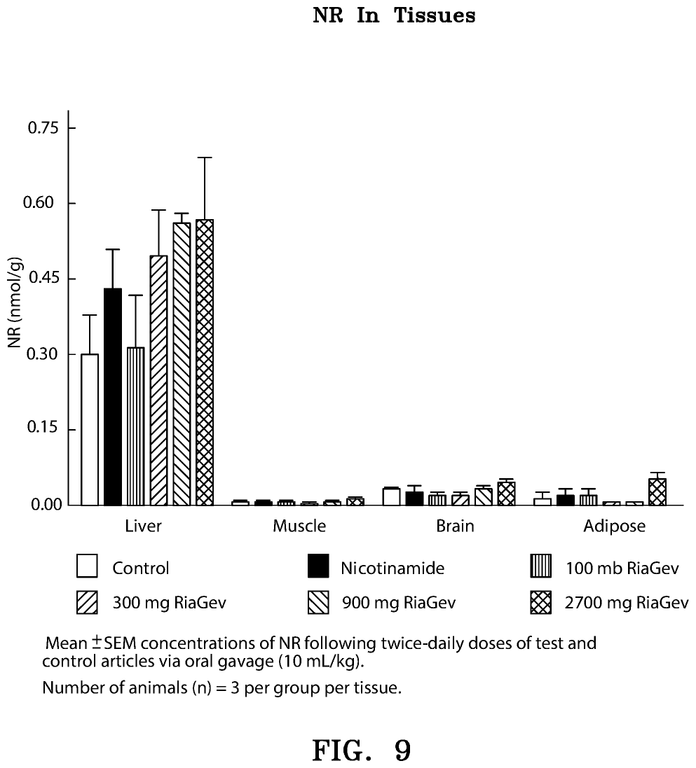

The NR concentrations in the liver, muscle, brain and adipose tissue are presented in FIG. 9. The highest levels of RiaGev derived NR were detected in the liver. The brain and adipose tissue had the next highest levels of NR, followed by the muscle tissue.

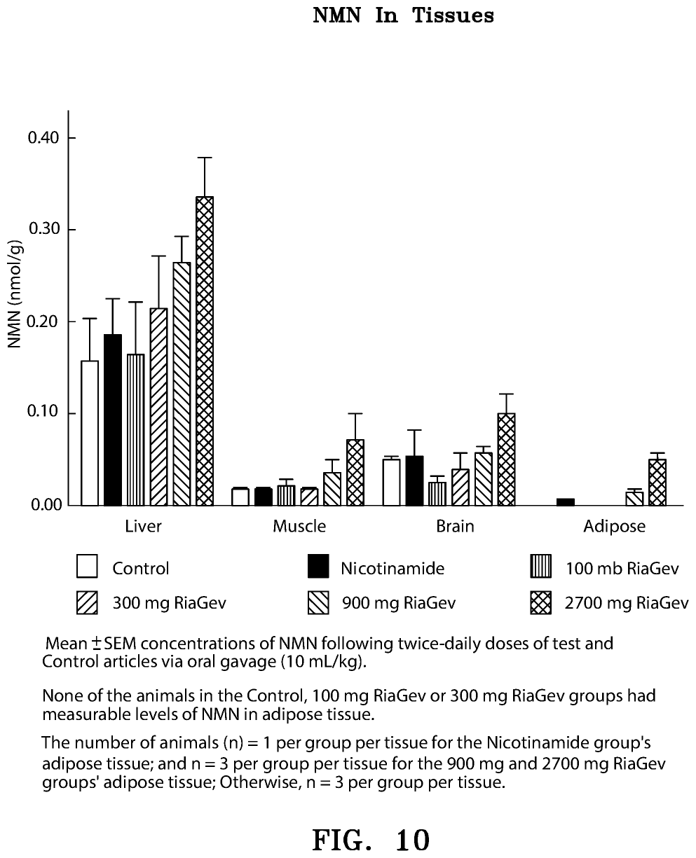

The NMN concentrations in the liver, muscle, brain and adipose tissue are presented in FIG. 10. The highest levels of RiaGev derived NMN were detected in the liver. The brain had the next highest levels of NMN, followed by the muscle, and then the adipose tissues. None of the animals in the control group, or in the 100 mg RiaGev or 300 mg RiaGev groups had measurable levels of NMN in adipose tissue.

### CONCLUSIONS AND DISCUSSION

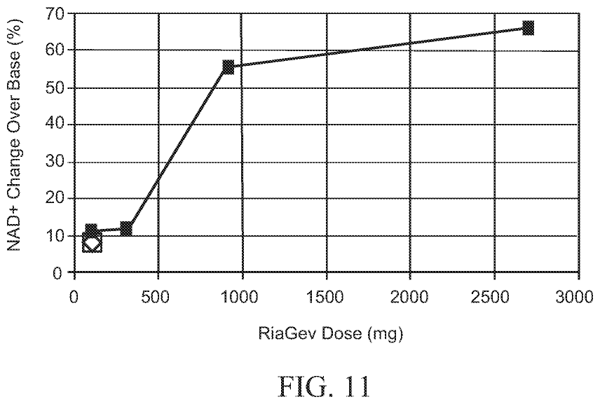

The experiment demonstrated that D-ribose combined with Nicotinamide (RiaGev) effectively elevates NAD metabolites, including NAD, MNN, and NR, levels in the body. For all the organs and tissue analyzed, a positive dose-response exists between RiaGev and NAD metabolites content. The 5-day time course experiment indicates that RiaGev derived NAD levels are cumulative and reached a plateau in the blood after 4 days (8 doses) of oral supplementation. The plateau concentration of NAD in blood versus dose is summarized in FIG. 11.

Referring to FIG. 11, the x-axis represents RiaGev doses and the y-axis shows blood NAD concentration change over its base (water control) level. It can be concluded that the most sensitive dose range is 300 mg/kg to 900 mg/kg BID, although RiaGev (D-ribose combined with Nicotinamide) increases NAD level at all dose levels tested (from 100 mg/kg to 2700 mg/kg BID). It is apparent that the Nicotinamide control is outside of the line, meaning D-ribose combined with Nicotinamide is much better than Nicotinamide by itself.

Comparing the final (5^{th} day) NAD concentration in the blood with concurrent NAD contents in the liver, muscle, and brain, it is noted that the blood and liver seems to be saturated with high dose RiaGev, while brain and muscle are not. This may reflect the fact that liver and blood are sites of NAD production and transfer, while muscle and brains are organs of NAD usage.

The pharmacodynamics of RiaGev is distinctly different from that of Vitamin B3 or NR alone. NR reaches its peak at about 8 hours after consumption. However, the 24-hour analysis indicated that RiaGev generates a broad elevation in NAD level without a significant peak. This implies a much more complex metabolism of RiaGev than other NAD boosting compounds which rely on a single enzyme such as NRK or NAMPT to get into a NAD synthetic pathway. Indeed, a closer look at the metabolic fate of the RiaGev main component, D-ribose, reveals that it may enter NAD synthetic pathways via multiple entrances.

D-ribose enhancing the efficiency of pyridine salvage via PRPP means that a lesser amount of pyridine is needed to achieve a response. One concern of large doses of Vitamin B3, including niacin, nicotinamide, or nicotinamide riboside, is the possible over taxation on detoxing capacity of the body, particularly methylation and hydroxylation capacities of the liver. The presence of D-ribose in RiaGev alleviates this concern. Therefore, D-ribose combined with Vitamin B3 not only is effective in increasing NAD levels, it also makes larger doses of Vitamin B3 supplementation safer to mammals, including humans.

1. A method of increasing NAD level in mammals by administering to the mammal an effective amount of D-ribose or D-ribose with Vitamin B3, where the Vitamin B3 includes any of: niacin, nicotinamide, nicotinamide riboside or nicotinamide mononucleotide.

2. The method of claim 1, wherein the effective amount is a ratio between 0.5:10 and 10:0.5 of Vitamin B3 to D-ribose.

3. The method of claim 1, wherein the effective amount is a ratio between 1:5 and 5:1 of Vitamin B3 to D-ribose.

4. The method of claim 1, wherein the effective amount is between 20 mg to 5400 mg per day.

5. The method of claim 1, wherein the effective amount is between 100 mg to 4000 mg per day.

6. The method of claim 1, wherein the effective amount is administered to the mammal at a time when the mammal is active or about to be active.

7. The method of claim 6, wherein the effective amount is administered to the mammal during daylight hours when the mammal is diurnal.

8. The method of claim 6, wherein the effective amount is administered in the morning.

9. The method of claim 6, wherein the effective amount is administered to the mammal during evening hours when the mammal is nocturnal.

10. A composition to be administered for increasing NAD level in mammals which comprises an effective amount of D-ribose or D-ribose with Vitamin B3, where the Vitamin B3 includes any of: niacin, nicotinamide, nicotinamide riboside or nicotinamide mononucleotide.

11. The composition of claim 10, wherein the effective amount is a ratio between 0.5:10 and 10:0.5 of Vitamin B3 to D-ribose.

12. The composition of claim 10, wherein the effective amount is a ratio between 1:5 and 5:1 of Vitamin B3 to D-ribose.

13. The composition of claim 10, wherein the effective amount is between 20 mg to 5400 mg per day.

14. The composition of claim 10, wherein the effective amount is between 100 mg to 4000 mg per day.
